(19) United States
(12) Patent Application Publication
(10) Pub. No.: US 2025/0367025 A1
(43) Pub. Date: Dec. 4, 2025

(54) SYSTEM AND METHOD FOR PAIN SUPPRESSION WITH COOLING

(71) Applicant: University of Central Florida Research Foundation, Inc., Orlando, FL (US)

(72) Inventors: Jazune Madas, Plantation, FL (US); Zixi Cheng, Oviedo, FL (US); Lei Zhai, Oviedo, FL (US); Swaminathan Rajaraman, Orlando, FL (US); Andre Childs, Orlando, FL (US)

(21) Appl. No.: 19/226,100

(22) Filed: Jun. 2, 2025

Related U.S. Application Data
(60) Provisional application No. 63/655,277, filed on Jun. 3, 2024.

(51) Int. Cl. A61F 7/00

(52) U.S. Cl. CPC A61F 7/0085; A61F 2007/0063; A61F 2007/0076

(57) ABSTRACT

A micro liquid thermal regulator (MLTR) utilizes closed-loop cooling and micro-channel technology to deliver targeted pain suppression. The system comprises a source of cooling liquid supplying the cooling liquid to a conduit that carries the cooling liquid to a neural cooling element. The neural cooling element has a cooling surface that is cooled by the cooling liquid supplied by the conduit, and is configured for placement in an area of nerve tissue in the organism such that the cooling surface cools the nerve tissue and reduces pain. Implanted onto ganglia, e.g., the dorsal root ganglia (DRG), the MLTR can reduce the temperature to as low as 10° C., effectively modulating internal molecular channel activity and reducing pain perception.

### RELATED APPLICATIONS

This application asserts the priority of U.S. provisional patent application Ser. number 63/655,277 filed on Jun. 3, 2024, which is herein incorporated by reference in its entirety.

This invention was made with Government support under Grant number NIH U01 NS113867-01 awarded by the National Institutes of Health. The Government has certain rights in this invention.

### FIELD OF THE INVENTION

This invention relates to the area of pain suppression, and more particularly to systems and methods using implantable devices for pain reduction or suppression.

### BACKGROUND OF THE INVENTION

According to a survey conducted by the National Center for Health Statistics (NCHS), a significant portion, approximately 20%, of American adults suffer from chronic pain. See Zelaya et al., Chronic Pain and High-impact Chronic Pain Among U.S. Adults, 2019. NCHS Data Brief, 390, 1-8 (2019). Furthermore, 8% of these individuals experience high-impact chronic pain, which significantly affects their quality of life, leads to opioid dependency, and negatively impacts mental health. High-impact chronic pain (HICP) significantly diminishes an individual's quality of life and daily functioning. The lack of adequate and safe non-opioid pain therapies highlights the need for alternative approaches.

One potential alternative therapy for pain management involves the application of cold temperatures. When a body system is exposed to temperatures below the normal range of 37° C., it disrupts the physiological processes within that system. See Sosnowski et al., “Hypothermia—Mechanism of action and pathophysiological changes in the human body”, Postepy Higieny I Medycyny Doswiadczalnej (Online), 69, 69-79 (2015), https://doi.org/10.5604/17322693.1136382. Lowering the temperature often leads to a slower physiological response. Recent studies have demonstrated the efficacy of cooling devices in blocking pain signals in peripheral nerves. See Reeder et al., “Soft, bioresorbable coolers for reversible conduction block of peripheral nerves”, Science, 377(6601), 109-115 (2022) https://doi.org/10.1126/science.ab18532.

These systems are general experimental systems, and they have drawbacks that need to be overcome, and processes and systems developed to be focused appropriately for pain management of various kinds in a practical treatment environment.

### SUMMARY OF THE INVENTION

It is accordingly an object of the present invention to provide apparatus and methods for delivering pain management to a patient, preferably a human, that avoids the drawbacks of the prior art.

According to an aspect of the invention, a system for pain reduction in an organism comprises a source of cooling liquid supplying the cooling liquid to a conduit. The conduit carries the cooling liquid to a neural cooling element. The neural cooling element has a cooling surface that is cooled by the cooling liquid supplied by the conduit. The neural cooling element is configured for placement in an area of nerve tissue in the organism such that the cooling surface cools the nerve tissue and reduces pain for the organism.

According to another aspect of the invention, a method of providing pain management comprises providing a system as described above and applying the neural cooling element of that system in a person with the cooling surface of the neural cooling element adjacent a ganglion of the person. The system is then caused to cool the cooling surface of the neural cooling element so as to provide temperature treatment to the ganglion.

Preferably, the temperature treatment comprises reducing the temperature of the ganglion below 20 degrees C.

In one aspect of the invention, the pain being managed is gastric pain, and the neural cooling element is applied to dorsal root ganglia of the person.

In a preferred embodiment of the invention, the applying of the neural cooling element is an implantation of the neural cooling element in the patient in proximity to the ganglion.

According to still another aspect of the invention, a chip configured for implantation in a human for delivering cooling treatment comprises first and second parts secured together. The first part has a top surface having an inlet opening and an outlet opening in it and first and second interior chambers extending downward from the openings and through a lower end of the first part. The first chamber communicates with the inlet opening and the second chamber communicates with the outlet opening. The second part has a plurality of coolant spaces in it open toward the first part and configured so that cooling fluid in the spaces causes cooling of a bottom surface of the second part. The first and second parts are configured such that a cooling fluid supplied through the inlet opening flows through the first chamber to the coolant spaces, and then flows from the coolant spaces to the second chamber and out of the first part through the outlet opening.

The first and second parts together form a cube having a dimension along each edge of 3 mm or less. The first part has a downward protrusion, and the second part has an upper recess receiving the downward protrusion enclosing an interior of the chip.

The second part has a portion through which the coolant spaces extend completely, and a metallic layer or aluminum tape overlies the coolant spaces and provides the bottom surface of the second part. The chambers of the first part are trapezoidal in horizontal cross section and each overlies all of the coolant spaces of the second part.

In the context of pain suppression, a compact micro-liquid thermo-regulator (MLTR) device utilizing 3D printing technology addresses the cooling of ganglia, especially spinal dorsal root ganglia (DRG; the origin of nociceptive axons in the stomach). The MLTR device is directly implanted into the DRG, enabling precise temperature control through microfluidic and thermo-regulation methods. The MLTR device effectively modulates nociceptive transmission by lowering the local temperature, providing a potential alternative to opioid-based pain suppression.

Other objects and advantages of the claimed apparatus and methods will become apparent from this specification.

### BRIEF DESCRIPTION OF THE DRAWINGS

FIG. 1 is a perspective view of a cooling system according to the invention.

FIG. 2 is a schematic diagram of the functional components of the MLTR coolant supply apparatus.

FIG. 3 is a schematic diagram of the electrical connections and control circuitry of the MLTR coolant supply apparatus.

FIG. 4 is a detailed side view of the MLTR chip.

FIG. 5 is a perspective view of the connection of the coolant tubing to the top part of the MLTR chip.

FIG. 6 is a perspective view of the coolant contact bottom side of the MLTR chip.

FIG. 7 is a cross-sectional view taken along line A-A of FIG. 4.

FIG. 8 is a cross-sectional view taken along line B-B of FIG. 4.

FIG. 9 is a cross-sectional view taken along line C-C of FIG. 4.

FIG. 10 is a perspective view of the bottom half of the MLTR chip showing the coolant contact surface on the bottom.

FIG. 11 is a diagram showing the flow of coolant through the MLTR chip.

FIG. 12 is a cross-sectional diagram showing flow of coolant in the bottom part of the MLTR chip.

FIG. 13 is a perspective view of the MLTR chip as fabricated without the coolant tubing attached.

FIG. 14 is a perspective view of the bottom side of the top part of the MLTR chip showing the manifold structure.

FIG. 15 is a perspective top view of the bottom part of the MLTR chip showing the cooling microchannels in the chip that cool the contact surface of the bottom of the chip.

FIG. 16 is a perspective view of the bottom of the lower part of the MLTR chip before the aluminum contact plate is applied.

FIG. 17 is a thermal image of an MLTR device with cooling turned on.

FIG. 18 is a graph showing the average temperature change obtained with the MLTR device exemplary of its operation.

FIG. 19 is a photograph of an alternate embodiment of a 3D printed bottom channel chip with 1000 μm scale bar for size that has five channels instead of four channels as in the previous embodiment.

### DETAILED DESCRIPTION

The micro liquid thermal regulator (MLTR) is an implantable medical device that utilizes closed-loop cooling and micro-channel technology to deliver targeted pain suppression. Implanted onto ganglia, e.g., the dorsal root ganglia (DRG), the MLTR can reduce the temperature to as low as 10° C., effectively modulating internal molecular channel activity and reducing pain perception. This offers a non-opioid alternative for pain management, providing precise and localized cooling to the source of pain transmission.

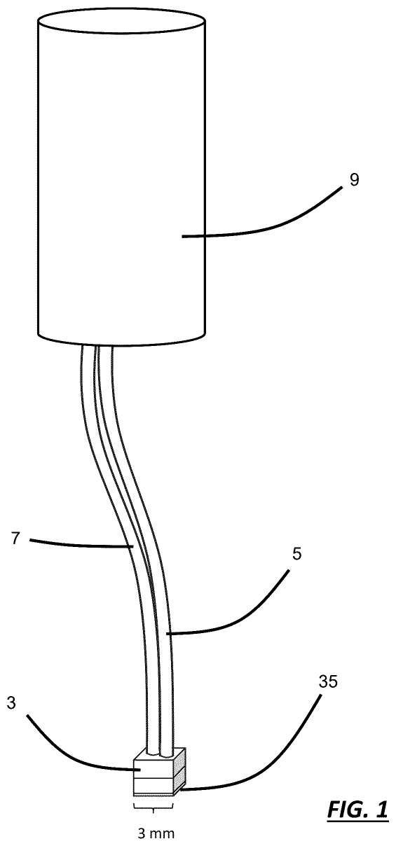

Referring to FIG. 1, a system 1 according to the invention comprises a neural cooling element in the form of an MLTR chip 3. Chip 3 is about 3 mm in size width, breadth and height, i.e., in all three dimensions, and is connected to two flexible tubes or conduits 5 and 7 that connect it fluidically to a control unit in housing 9. The conduits carrying the cooling fluid are preferably 1/16-inch diameter tubing.

The control unit housing 9 encloses electronic circuitry that supplies cooling fluid through the first conduit 5 to the chip 3 and draws back the cooling fluid that has cooled and been heated by the chip 3 through the second conduit 7. The housing and control unit incorporates a closed loop cooling mechanism, providing unique advantages, including portability, further enhancing its practicality in pain management applications.

The cooling fluid supplied to the chip produces cooling of a metallic cooling contact surface of the aluminum bottom plate or layer 35 of the chip 3 down to temperatures below 20 degrees C., and potentially as low as 15 degrees C. or even 10 degrees C., which makes the MLTR system and method of the invention suitable as a non-opioid alternative for pain suppression. Localized cooling can effectively modulate pain signals transmitted to the central nervous system (CNS). By achieving temperatures lower than the physiological norm of 37° C., the MLTR can influence the underlying physiological processes involved in pain transmission. Its miniature design makes the MLTR suitable for implantation into, e.g., the dorsal root ganglia (DRG) to impact on pain signaling to the brain.

The dorsal root ganglia (DRG) serves as a critical point for pain signal mediation within the body. By surgically implanting the MLTR chip 3 onto the DRG, the device can cool that area, leading to the closure of internal ion channels such as TRPV1 that are responsible for transmitting pain signals to the CNS. The compact size of the MLTR further enables its application in various anatomical regions, allowing efficacy in different temperature ranges and pain conditions, providing targeted pain relief through precise temperature modulation to reduce pain perception.

In the specific area of chronic visceral gut pain management, the micro-liquid thermo-regulator (MLTR) system particularly offers an alternative to opioid-based therapies. When the gastrointestinal system is affected by HICP, it can be particularly debilitating due to the brain-gut connection. The gut perceives pain stimuli through nociceptive afferent fibers originating from the spinal dorsal root ganglia and vagal nodose ganglia, which transmit pain signals locally or to the central nervous system. Addressing pain at the DRG location can be effective at pain management for such cases.

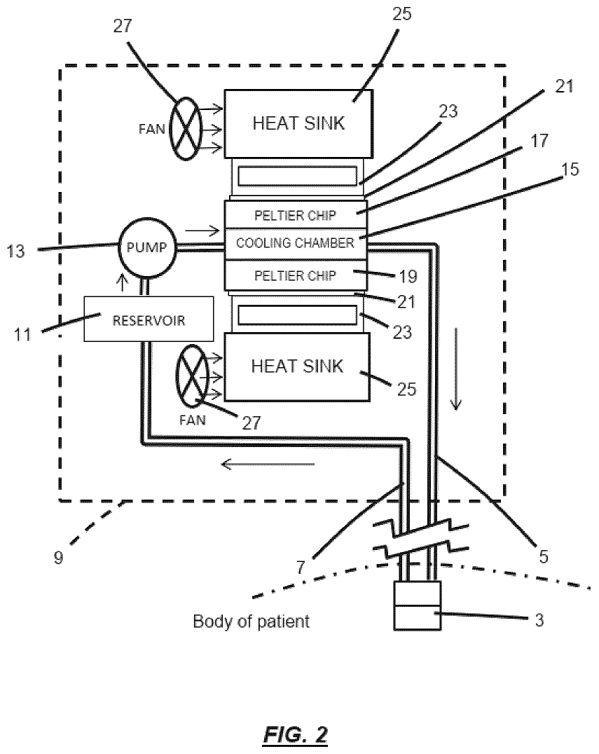

Referring to FIG. 2, the micro liquid thermal regulator (MLTR) system comprises several components, including a cooling chamber, water pump, MLTR, and thermocouple. The primary function of this configuration is to cool the coolant before it reaches the MLTR chip 3.

Housing 9 supports in it a reservoir 11 connected to a pump 13 that connects to a cooling chamber 15. The tube 5 carries the cooling fluid from the cooling chamber 15 to the chip 3, through which it flows, cooling the bottom surface of the chip, which is implanted in an organism, usually a human patient, in an appropriate location for temperature therapy, especially a ganglion or DRG. The fluid then returns from the chip 3 through conduit 7 to reservoir 11, from which it is drawn by pump 13 and supplied back to the cooling chamber 15 for further cooling. This group of components provides a closed system with the same cooling fluid re-used continually, and driven by the pump 13.

The cooling chamber 15 is cooled by two Peltier chips 17 and 19 secured to both sides of the chamber 15 and cooling it and its contents. Heat extracted is transmitted through thermal interface material 21 to heat pipes 23 that carry the heat to heat sinks 25. Fans 27 blow cooler air from the ambient environment through the housing so as to expel the heat from the heat sinks 25.

Peltier chips and associated heat pipes and heat sinks are well known in the art and may be purchased as integrated units. One such unit used in development of the present system is sold by amazon.com under the brand name Hilitand with the description DC 12V Thermoelectric Cooler Peltier System Semiconductor Refrigeration Water Chiller Cooling Device. However, a wide variety of systems of this sort are readily available as off-the-shelf units and can be used in the present system.

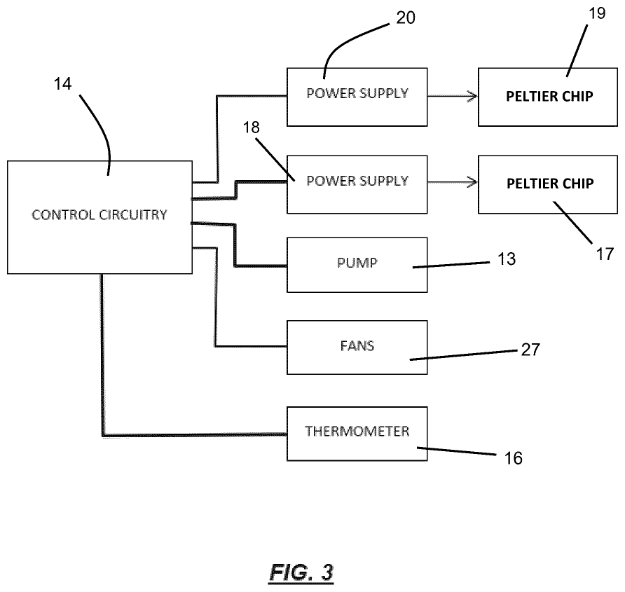

FIG. 3 illustrates the general electrical arrangement of the system. Control circuitry 14 is connected with power supplies 18 and 20 for the Peltier chips 17 and 19, the pump 13, and the fans 27. The control circuitry operates the fans, pump and Peltier chips so as to ensure a suitable flow of adequately cooled cooling fluid to the chip 3, such that cooling to a predetermined temperature level set through the control circuitry.

To monitor the thermo-regulation operations of the MLTR, thermocouple or other temperature sensor or micro-thermometer 16 is securely attached to the bottom of the chip 3, and is also electrically connected with the control circuitry 14. This thermometer 16 is operatively connected with the cooling surface of the chip 3, and sends back electrical signals along a conductor extending alongside the conduits 5 and 7, which signals correspond to the temperature of the cooling surface 35 applied in the patient, to enable real-time temperature monitoring and to provide assessment of the effectiveness of the cooling. The assessment of the device's operation to cool the skin, muscle, and dorsal root ganglia (DRG) locally is accomplished by placing the micro thermometer between the target area of the patient's tissue and the MLTR, and monitoring the local temperature reduction. The MLTR is used to lower the temperature to at least as low as 20° C., as this threshold should impact the DRG's pain transmission capacity.

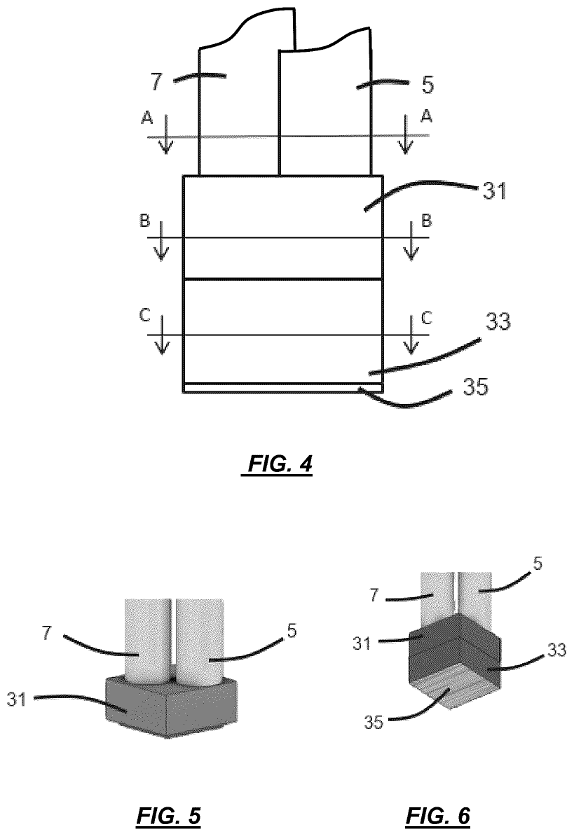

FIGS. 4 to 16 show details of the design of the MLTR chip 3 along with the orientation of the micro-manifold designs.

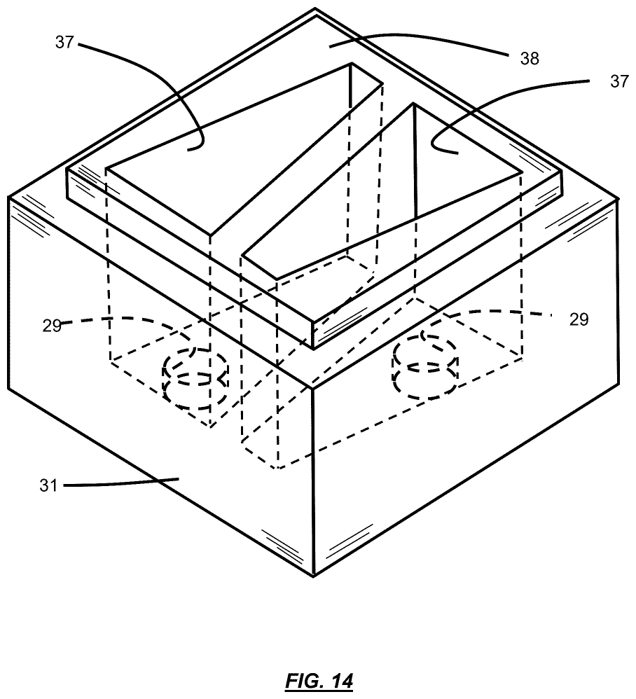

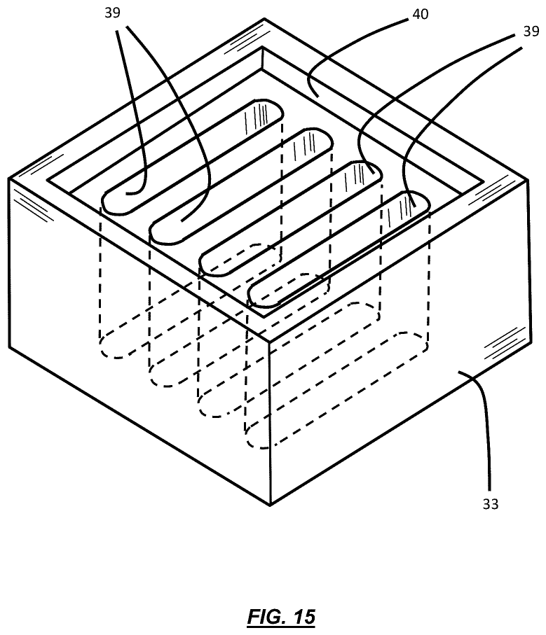

Referring to FIGS. 4, 6, and 13 the MLTR chip includes two discrete parts 31 and 33 that combine into a cubic article with a bottom side and top side. The top part 31 has dimensions of 3 mm×3 mm×1.7 mm, and the bottom part 33 dimensions are 3 mm×3 mm×1.5 mm. As best seen in FIG. 14, the top part (shown upside down, with its attachment side up) has in its center a raised 2.4 mm square projection 38 with a height of 0.2 mm that mates with a corresponding square recess 40 in bottom part 33 (FIG. 15), with the two parts 31 and 33 sealed and securely attached to each other by a sealing adhesive.

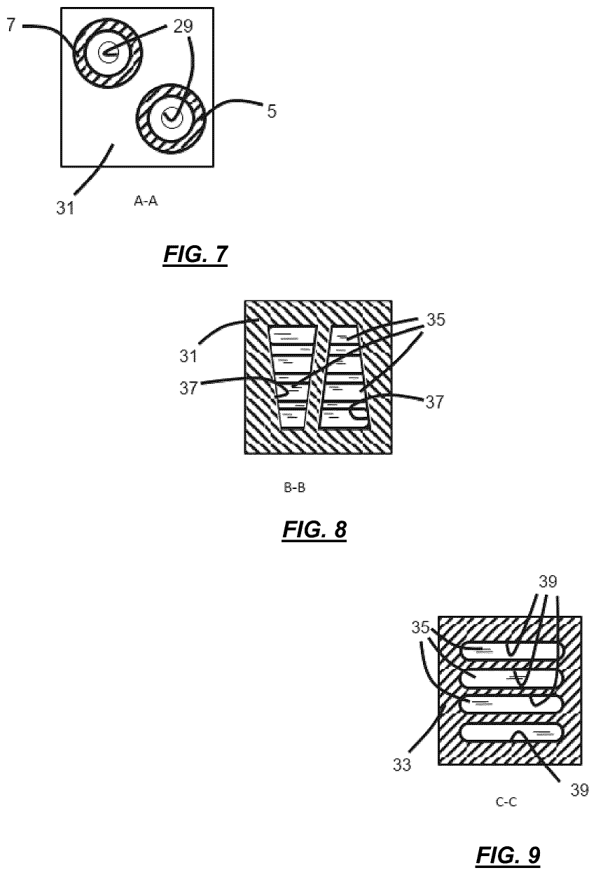

FIG. 5 shows the MLTR chip with the connection of conduits 5 and 7. Those conduits each are secured in a fluid-tight way over a respective one of openings 29 (see section A-A in FIG. 7 and FIG. 13) in the top part 31 so as to feed the cooling fluid to the interior space of the chip 3.

The openings 29 in top part 31 communicate with manifold channels in the top part which are two trapezoidal spaces 37 that face opposite one another. These trapezoid spaces 37 may be seen in horizonal cross section in FIG. 8, and also from the bottom view of the top part 33 seen in FIG. 14. Vertical cross sections of those trapezoidal manifold spaces are also seen in FIG. 11. The manifold spaces 37 create a hollow interior space inside of the top part 31 going 1.5 mm deep leaving a 0.2 mm wall on the top of the part through which the openings 29 extend and to the top of which the conduits 5 and 7 are secured. The openings 29 are each a 0.4 mm hole or opening in the top wall of top part 31 (on the bottom in FIG. 14 and on the top in FIG. 13). Each opening 29 is located so that it extends to the respective trapezoidal space in a wider portion of the trapezoid cross-sectional shape.

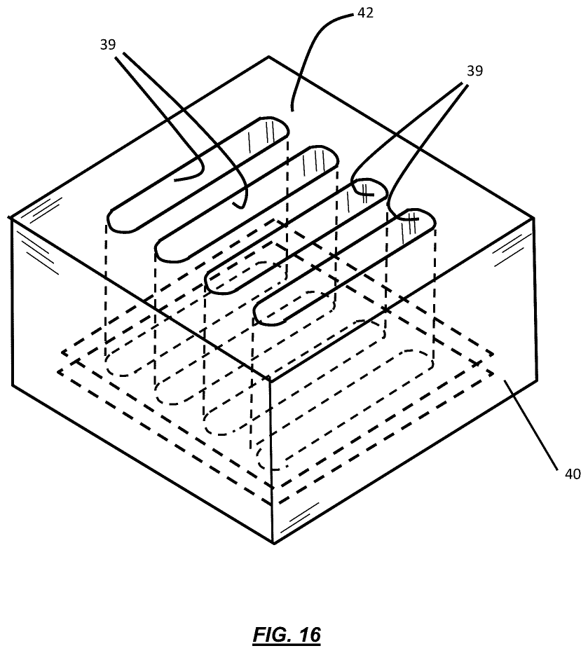

The bottom part 33 is seen in detail in FIGS. 15 and 16. The upward facing square recess 40 of bottom part fits into the protrusion in top part 31. Bottom part 33 has micro channels 39 extending through it that are configured to receive cooling fluid from the trapezoidal spaces of part 31. The micro-channels are 2 mm in length with a width of 300 μm and four of them are spaced 258 μm apart of one another. The channels are also spaced so that they are 0.2 mm away from the wall. As the bottom part 33 is fabricated, the channels 39 also each create a passage going completely through to the opposite (bottom) side of the bottom part 33.

The openings 29 each communicate with respective interior trapezoidal spaces 37, and those trapezoidal spaces 37 are open at their lower ends toward the bottom part 33 and the micro-channels. Each of the trapezoidal manifold spaces runs perpendicular to the microchannels and communicates with all of the micro-channels 39, as may be seen in FIG. 8, in which the micro-channels 39 are visible in the cross-sectional view through the trapezoidal spaces.

The bottom of bottom part 33 and the lower ends of the micro-channel 39 passages are covered and sealed by metallic wall or layer 35. The metallic layer is preferably metal in the form of aluminum tape adhered to part 33. The design allows minimal water pressure but a high liquid throughput, creating a highly efficient thermoregulation effect.

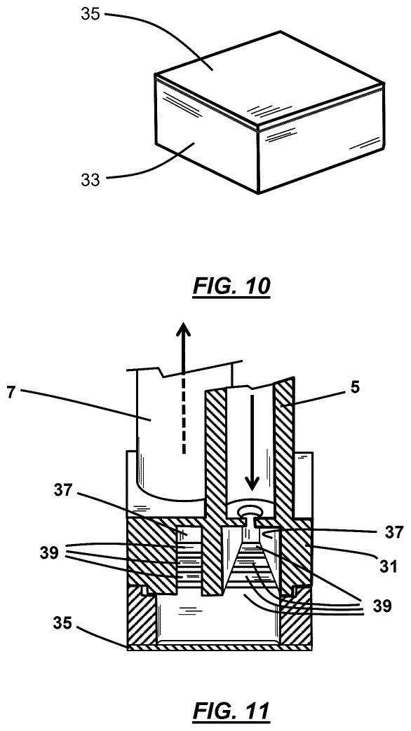

Metallic layer 35 is visible in the cross-sectional views of FIGS. 8 and 9 through the micro-channels 39, and also may be seen overlying the bottom surface of part 33 in FIG. 10, where the bottom part 33 is shown upside down. The outer surface of the metallic layer 35 is the cooling contact surface for the neural cooling element, i.e., the MLTR the chip 3.

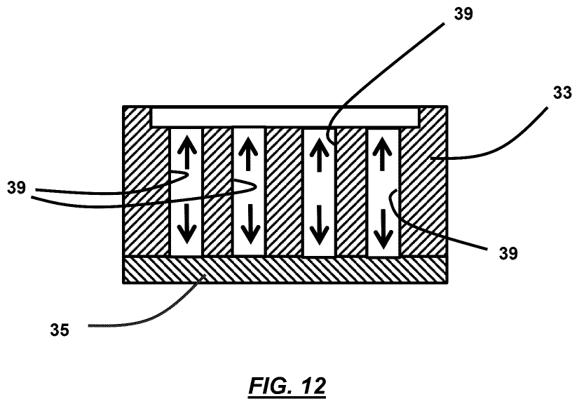

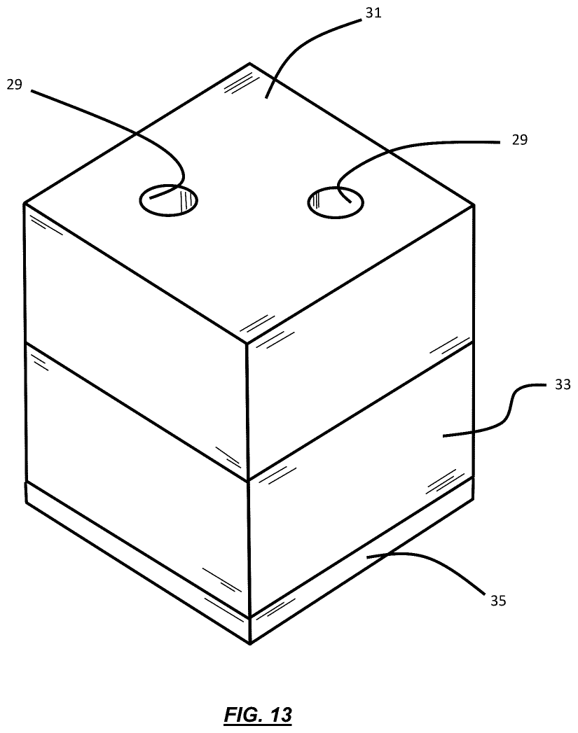

In operation, as best shown in FIG. 11, the cooling fluid from conduit 5 flows into one of the manifold spaces 37. The bottom of the trapezoidal space 37 connects with all of the micro-channels 39, and the cooling fluid flows into the microchannels, illustrated by the downward arrows of flow seen in the detail view of bottom part 33 seen in FIG. 12, then flowing along the metallic wall or layer 35. During that flow in the micro-channels 39, the aluminum bottom layer 35 is cooled, which cools the adjacent tissue or ganglia of the patient receiving treatment. The coolant fluid, heated to a degree by heat from the tissue of the treated patient, then flows upward (the upward arrows of FIG. 12) into the other trapezoidal manifold 37, out of which the cooling fluid flows or is drawn via conduit 7 (FIG. 11).

MLTR chip parts 31 and 33 are preferably made using 3D printing technology, which allows for the rapid and cost-effective fabrication of the MLTR, while enabling iterative design adjustments to enhance efficiency. An efficient micro-manifold design within a millimeter-scale device that can be implanted is fabricated using digital light processing (DLP) 3D printing (Asiga Max XUV 27) with Formlabs clear resin. The design allows minimal water pressure but a high liquid throughput, creating a highly efficient thermoregulation effect. The design also incorporates a closed-loop cooling system, which allows for heat dispersion outside of the treated person and requires only a power input to keep the system running. The device offers a microscale, long-lasting, implantable, and closed-loop thermal regulator applicable to continuous thermal effects on physiological processes toward chronic pain applications. The utilization of 3D printing in the construction of the MLTR brings several advantages, including ease of fabrication and design flexibility. A 3D printed micro liquid thermal regulator or MLTR can be used for in-vivo thermoregulation.

The design of parts 31 and 33 is exported as an stl file and then imported into the Asiga composer software. Optimized settings are input for 3D printing each side using the Asiga Max XUV 27 with Formlabs clear resin. After printing both parts 31 and 33, they are taken off the stage and cleaned using 90% isopropyl alcohol (IPA). Support structures are snipped off and sanded down until flush. The parts are then cleaned again, loaded into a 50 ml centrifuge tube with 90% IPA, and put into a sonication bath for 5 minutes. The parts are then left to dry until all of the IPA has evaporated.

On top part 31 of the device, pieces of 1/16 inch tubing are adhered onto each of the inlet and outlet openings 29 using the same resin as the printed parts and spot curing with a handheld UV flashlight. The bottom of the microchannels features an aluminum layer that enables efficient thermal transfer, and a thermocouple is placed between the aluminum layer and the target area using thermal paste to measure temperature changes accurately. The bottom part 33 then has a 3 mm×3 mm square of aluminum tape adhered to the flat area over the open micro channels 39. This tape is pressed in place for 5 minutes under the constant pressure of a clamp.

The pieces 31 and 33 are then aligned, pressed together, and adhered to each other using the same resin as the printing and spot curing with UV light. Multiple layers of resin are used to ensure a water-tight seal for the device 3 and also around the outer area of the aluminum layer 35. Finally, the device 3 is put into a Formlab curing chamber for 15 minutes to ensure all resin pieces have been fully cured. A syringe filled with deionized DI water is then pushed down the inlet tube to check for any leak.

The cooling unit of the MLTR system is assembled from its various components, described above, including two 12V Peltier chips, a heat sink, thermal interface material (TIM), fans, a cooling chamber, and heating pipes. TIM is applied to both sides of the cooling chamber to assemble the core cooling unit. The Peltier chips are then carefully positioned to sandwich the cooling chamber, ensuring that TIM is also applied to the other side of the chips. A premade heat sink, which already has the heating pipes attached, is then connected to the other side of the Peltier chips, ensuring proper contact and thermal conductivity. Fans are positioned to facilitate the flow of cold air into one side of the unit while allowing hot air to exit from the other side.

Each Peltier device requires its own power source, with one set requiring 120 W and the other requiring 72 W. A segment of ¼ inch tubing is immersed in a 50/50 Antifreeze coolant mixture. This tubing is then connected to a Masterflex Easy-load 7518 pump allowing the coolant to circulate. Another segment of ¼ inch tubing is attached from the pump to the inlet of the cooling chamber. An adapter is used to connect the outlet tubing of the cooling chamber to the inlet tubing of the MLTR. The outlet tubing of the MLTR is then routed back into the reservoir tank. This setup creates a closed cooling system, as the coolant is returned to the reservoir, ensuring a continuous cooling cycle.

The coolant fluid or liquid is preferably water-based fluid with antifreeze, which may be any admixture to water that prevents ice formation in the cooling system. A variety of anti-freeze solutions exist and are well known in the art. One suitable antifreeze liquid is Peak Premium 50/50 antifreeze sold by Old World Industries, Northbrook, Illinois.

The MLTR's control unit current design incorporates two Peltier chips, necessitating a power draw of 192 W. As a result, two power sources are required to operate the cooling system at its maximum temperature reduction. This additional power requirement may contribute to the overall size of the cooling system, potentially making it less portable. However, in an alternate embodiment, the system may employ smaller Peltier chips, or implement designs that can achieve an acceptable cooling effect with a reduction power consumption.

Alternatively, the MLTR system may depart from the closed-loop cooling system altogether, and instead utilize stored cryogenic liquids such as nitrogen to cool the cooling surface of the MLTR neural cooling element or chip.

An advantageous feature of the system is that it features a close loop cooling system as the coolant is recycled from the MLTR to the cooling chamber.

The essential cooling component of the MLTR, utilizes Peltier chips in direct contact with a cooling chamber, creating a closed system enabling rapid temperature reduction of the coolant before it reaches the chip. This configuration ensures that the device can achieve and maintain low temperatures over an extended period, while the micro-thermometer is attached to the target area to monitor the effectiveness of the cooling process.

The 3D printing capability of the MLTR enables efficient fabrication and easy customization, making it a versatile and adaptable device. With its closed loop cooling system, the MLTR operates independently, requiring only power for functionality, which enhances its potential for portability. This portability allows the device to provide localized cooling to internal targets after implantation, offering a promising solution for pain management.

The MLTR incorporates a closed loop cooling system instead of relying on natural cold gases or liquids. This eliminates the need for regular refilling and offers enhanced control over temperature regulation. By adjusting the power supplied to the cooling chamber, the MLTR allows for precise modulation of temperature, providing a customizable and efficient cooling solution. The combination of 3D printing technology and closed loop cooling allows the MLTR to offer convenience, temperature control, and adaptability for pain management applications.

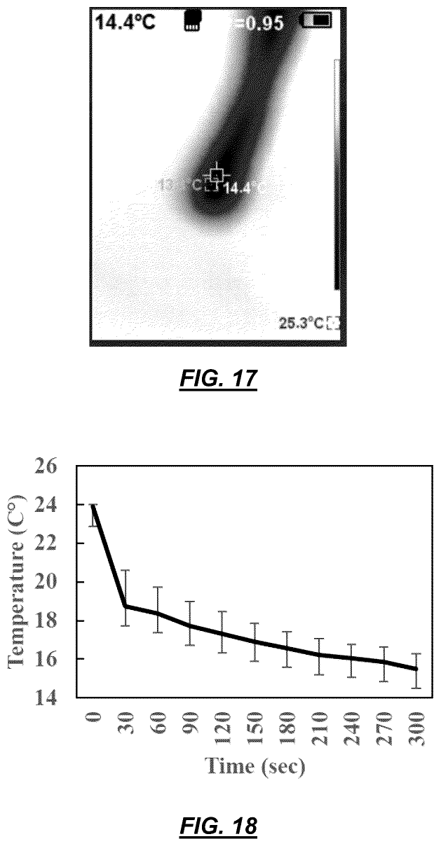

FIG. 17 is a thermal image depicting an MLTR unit reaching 13.8° C. from a side view analysis of the device. An internal temperature of 13.8° C. is indicated by the dark color, i.e., the darkest center part of the image, and an outside temperature of 14.4° C. indicated at the boundary of the area, as well as a surrounding environment of the room temperature of 25.3° C. indicated in the white color.

Table 1 shows the results of ten exemplary activations of the MLTR over a period of 5 minutes, showing the reduction from room temperature. The results are summarized in FIG. 18, which shows the average temperature change when the device is powered on.

TABLE 1
Thermo-Regulation 5-min Test using the MLTR

| | Temperature Change (° C.) | | | | | | | | | |
|---|---|---|---|---|---|---|---|---|---|---|
| Time | Trial | Trial | Trial | Trial | Trial | Trial | Trial | Trial | Trial | Trial |
| (sec) | 1 | 2 | 3 | 4 | 5 | 6 | 7 | 8 | 9 | 10 |
| 0 | 23.8 | 24 | 24 | 23.9 | 23.7 | 24 | 23.8 | 23.9 | 23.7 | 24 |
| 30 | 22.1 | 20.9 | 18.9 | 20.4 | 18.1 | 17.9 | 18.5 | 16.2 | 16.8 | 17.5 |
| 60 | 20.7 | 20 | 18.8 | 19.5 | 18 | 17.9 | 17.8 | 16.3 | 17.4 | 17.2 |
| 90 | 19.5 | 19.7 | 18.2 | 18.4 | 17.5 | 17.4 | 17 | 15.7 | 16.9 | 16.8 |
| 120 | 18.9 | 19.3 | 18 | 17.5 | 17.2 | 16.8 | 16.3 | 15.5 | 16.8 | 16.7 |
| 150 | 17.7 | 18.5 | 17.9 | 17.3 | 17 | 16.6 | 16 | 15.4 | 16.3 | 16.2 |
| 180 | 17.1 | 18.2 | 17.1 | 16.9 | 16.8 | 16.2 | 16 | 15.2 | 16.1 | 15.9 |
| 210 | 16.7 | 17.3 | 17.3 | 16.7 | 16.4 | 16.2 | 15.4 | 14.7 | 15.7 | 15.6 |
| 240 | 16.6 | 17 | 16.6 | 16.1 | 16.3 | 16.4 | 15.6 | 14.7 | 15.9 | 15.2 |
| 270 | 16.3 | 16.7 | 16.9 | 16.4 | 16.1 | 16 | 15 | 14.7 | 15.3 | 15.1 |
| 300 | 16.2 | 16.6 | 16.3 | 15.5 | 15.9 | 15.7 | 14.5 | 14.5 | 14.9 | 14.7 |

The example began by activating the pump to initiate coolant circulation throughout the system. The initial temperature reading was recorded without the MLTR to establish a baseline. Then, the MLTR was installed, and the temperature reading was noted prior to activating the cooling system. Once the cooling system was turned on, temperature measurements were taken at intervals of 30 seconds for a duration of 5 minutes. The test was repeated 10 times showing the cooling capability.

The initial temperature recorded before activating the MLTR was 23.88° C. on average. The temperature gradually decreased throughout the 300-second trial period, reaching an average temperature of 15.48° C. This corresponds to an average temperature drop of 8.4° C. Over the course of room temperature tests (n=10), the device was able to lower the temperature from 25.3° C. to 14.4° C. An average of 8.4° C. temperature drop was calculated.

A longer test was also performed where the MLTR was allowed to stay on until it reached 12° C., where it stayed.

By achieving temperatures lower than the physiological norm of 37° C., the MLTR can influence the underlying physiological processes involved in pain transmission. The results show that the MLTR can reduce temperature near the spine affecting the DRG, skin and muscle organelles.

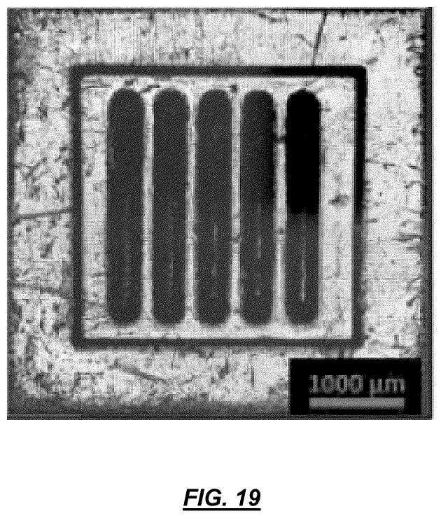

FIG. 19 shows another embodiment of the MLTR chip that is similar to the chip described above, but is provided with five micro-channels, rather than four. The number of micro-channels employed in the invention may vary.

The terms used herein should be understood to be terms of description rather than limitation, as those of skill in the art will be able to make modifications to the description herein without departing from the spirit of the invention.

What is claimed is:

1. A system for pain reduction in an organism, said system comprising:
a source of cooling liquid supplying the cooling liquid to a conduit;
a conduit carrying the cooling liquid to a neural cooling element;
the neural cooling element having a cooling surface that is cooled by the cooling liquid supplied by the conduit;
the neural cooling element being configured for placement in an area of nerve tissue in the organism such that the cooling surface cools the nerve tissue and reduces pain for the organism.

2. The system of claim 1, wherein the neural cooling element is a unit having
an inlet connected with the conduit and receiving the cooling liquid therefrom to go to the neural cooling element, and
an outlet connected with a second conduit receiving for the cooling liquid after passing through the neural cooling element;
the neural cooling element having an interior space communicating with the inlet and outlet such that the cooling liquid flows therethrough and cools the cooling surface.

3. The system of claim 2, wherein the second conduit connects the outlet to the source;
the source having a pump causing flow of the cooling liquid therethrough, said source receiving the cooling liquid back from the neural cooling element, cooling the cooling liquid returned, and then transmitting the cooled cooling liquid to the neural cooling element.

4. The system of claim 3, wherein the source includes control circuitry connected with a temperature sensor associated with the neural cooling element, said control circuitry controlling operation of the source so as to maintain a preselected temperature of tissue in contact with the cooling surface of the neutral cooling element.

5. The system of claim 4, wherein the source includes a reservoir storing the cooling liquid and a cooling chamber cooled by at least one Peltier chip connected with a heat sink.

6. The system of claim 2, wherein the source comprises a housing having therein
a reservoir holding cooling liquid and connected so as to receive cooling liquid from the second conduit;
a pump connected with the reservoir and drawing therefrom cooling liquid and supplying the cooling liquid to a cooling chamber;
the cooling chamber being in communication with the first conduit such that the cooling liquid is cycled by the pump through the cooling chamber, then to the first conduit, then to the inlet opening of the chip, then through the cooling spaces of the chip, then through the outlet opening of the chip, then to the reservoir, then back to the pump;
the cooling chamber having operatively connected therewith two Peltier chips that are both powered so as to cool the cooling chamber and the cooling liquid therein;
the Peltier chips each being connected thermally through thermal connecting material and a heat pipe to a heat sink;
the heat sink being exposed to airflow by one or more fans drawing cool air from the environment and through the housing;
each of the Peltier chips being powered by a respective power supply; and
a temperature detector or thermocouple connected with the metallic layer and communicating electrically with control circuitry so as to transmit thereto data corresponding to a temperature of the cooling surface of the chip, said control circuitry controlling operation of the source so as to cool the cooling surface to below a predetermined temperature.

7. The system of claim 3, wherein the neural cooling element includes a body that is formed of non-metallic material supporting a metallic wall portion, said cooling surface being on the metallic wall portion;
the metallic wall portion being at least partly exposed on a side thereof opposite to the cooling surface to the interior space in the neural cooling element so as to contact the cooling liquid in the interior space and to be cooled thereby.

8. The system of claim 3 wherein the interior space is divided into a plurality of parallel cooling channels through which the cooling liquid flows and cools the cooling surface.

9. The system of claim 8, wherein the inlet has a manifold receiving the cooling fluid and communicating with cooling channels so as to supply the cooling liquid from the inlet thereto.

10. The system of claim 9, wherein the outlet has a manifold communicating with the cooling channels so that cooling fluid passes through the neural cooling element by passing through the manifold of the inlet, through all of the cooling channels, through the manifold of the outlet and out of the neural cooling element.

11. The system of claim 10, wherein cooling element is formed in two parts, one of the parts having openings for the inlet and outlet and the manifolds therein, and
the other of the parts having the cooling channels therein extending completely therethrough, and
a wall overlying the cooling channels and being cooled by cooling liquid therein, the cooling surface being on a side of the wall opposite to the cooling channels.

12. The system of claim 1, wherein the wall is formed of metal.

13. The system of claim 1, wherein the cooling surface has a maximum dimension of 2.5 to 3.5 mm.

14. A method of providing pain management, said method comprising
providing a system according to claim 1;
applying the neural cooling element thereof in a person with the cooling surface of the neural cooling element adjacent a ganglion of the person; and
causing the system to cool the cooling surface of the neural cooling element so as to provide temperature treatment to the ganglion.

15. The method of claim 14, wherein the temperature treatment comprises reducing the temperature of the ganglion below 20 degrees C.

16. The method of claim 14, wherein the pain being managed is gastric pain, and the neural cooling element is applied to dorsal root ganglia of the person.

17. The system of claim 14, wherein the applying of the neural cooling element is an implantation of the neural cooling element in the patient in proximity to the ganglion.

18. A chip configured for implantation in a human for delivering cooling treatment, said chip comprising:
first and second parts secured together;
the first part having a top surface having an inlet opening and an outlet opening therein,
the first part having first and second interior chambers extending downward from the openings and through a lower end of the first part, said first chamber communicating with the inlet opening and the second chamber communicating with the outlet opening;
the second part having a plurality of coolant spaces therein open toward the first part and configured so that cooling fluid in said spaces causes cooling of a bottom surface of the second part;
the first and second parts being configured such that a cooling fluid supplied through the inlet opening flows through the first chamber to the coolant spaces, and then flows from the coolant spaces to the second chamber and out of the first part through the outlet opening;
the first and second parts together forming a cube having a dimension along each edge of 3 mm or less, said first part having a downward protrusion and the second part having an upper recess receiving the downward protrusion enclosing an interior of the chip;
the second part having a portion through which the coolant spaces extend completely, and a metallic layer or aluminum tape overlying the coolant spaces and providing the bottom surface of the second part; and
the chambers of the first part being trapezoidal in horizontal cross section and each overlying all of the coolant spaces of the second part.

19. The chip according to claim 18, wherein the first and second parts are formed by 3D printing of a resin material.
